(19) United States
(12) Patent Application Publication
(10) Pub. No.: US 2009/0264981 A1
(43) Pub. Date: Oct. 22, 2009

(54) METHOD OF USING OPTICAL COHERENCE TOMOGRAPHY (OCT) IN SURGERY

(75) Inventors: Robert Poston, Newton, MA (US); Cha-Min Tang, Radnor, PA (US)

Correspondence Address: CASTELLANO PLLC, P.O. Box 1555, Great Falls, VA, 22066, US

(73) Assignee: UNIVERSITY OF MARYLAND, BALTIMORE

(21) Appl. No.: 12/303,716

(22) PCT Filed: Jun. 7, 2007
(86) PCT No.: PCT/US07/13408

§ 371 Date: Feb. 4, 2009

Related U.S. Application Data
(60) Provisional application No. 60/811,916, filed on Jun. 7, 2006.

(51) Int. Cl. A61F 2/06; A61B 6/00

(52) U.S. Cl. 623/1.13; 600/476

(57) ABSTRACT

Methods comprising the use of optical coherence tomography to examine a harvested conduit for its suitability for grafting, to examine a candidate conduit prior to harvesting for coronary artery bypass graftings to assess the quality of an anastomosis, to assess the quality of the suturing of a small vessel or duct with a suture coated with a contrast agent, and to detect coronary artery disease in a harvested donor heart prior to transplantation.

### CROSS-REFERENCE TO RELATED APPLICATION

This application claims priority to co-pending U.S. provisional patent application no. 60/811,916, which was filed on Jun. 7, 2006, and which is hereby incorporated by reference in its entirety.

### TECHNICAL FIELD

The present disclosure relates to OCT, vascular imaging, and surgery.

### BACKGROUND

Between 300,000 and 400,000 coronary artery bypass grafts (CABG) are performed annually in the U.S. Restenosis occurs in about 5-15% of the newly grafted conduits. It has long been suspected that this complication is due to injury of the inside of the vessel during harvesting. Unfortunately, there currently is no practical way of detecting this injury prior to grafting.

It is an object of the present disclosure to provide a method of examining a harvested conduit and identifying a portion thereof, which is less likely to result in premature stenosis after grafting. This and other objects and advantages, as well as inventive features, will be apparent from the detailed description provided herein.

### BRIEF SUMMARY

Provided is a method of examining a harvested conduit. The method comprises using OCT to identify a portion of the harvested conduit as suitable for grafting.

Also provided is a method of examining a candidate conduit prior to harvesting for CABG. The method comprises using OCT to identify a portion of the candidate conduit as suitable for harvesting and subsequent grafting.

Still also provided is a method of assessing the quality of an anastomosis. The method comprises using OCT and an imaging catheter to generate serial images of the anastomosis from inside of the anastomosis, generating a three-dimensional reconstruction of the anastomosis, examining the three-dimensional reconstruction of the anastomosis to assess the quality of the anastomosis, repairing the anastomosis as needed, and closing the surgical incision used to suture the anastomosis.

Further provided is a method of assessing the quality of the suturing of a small vessel or duct. The method comprises using OCT and an imaging catheter to generate serial images of the site of suturing from inside the small vessel or duct, generating a three-dimensional reconstruction of the site, examining the three-dimensional reconstruction of the site to assess the quality of the suturing, and repairing the suturing as needed. The suture is coated with a contrast agent prior to suturing.

Still further provided is a method of detecting coronary artery disease in a harvested donor heart prior to transplantation. The method comprises scanning the harvested donor heart with OCT to generate serial images of the heart and examining the images for signs of disease, whereupon coronary artery disease can be detected in a harvested donor heart prior to transplantation.

### DETAILED DESCRIPTION

Provided is a method of examining a harvest conduit (i.e., ex vivo). By “conduit” is meant any blood vessel, which can be harvested and subsequently grafted. Non-limiting examples of such a conduit include a radial artery (RA) and a saphenous vein (SV).

The method comprises using OCT to identify a portion of the harvested conduit as suitable for grafting. A cannula can be placed inside of the conduit, and the OCT probe can be inserted into the cannula. Concurrent gentle Hank's balanced salt solution (HBSS) infusion, such as by means of a Y-connector connected to the cannula, straightens the conduit for optimal imaging. OCT enables high-resolution, sub-surface, micro-structural images to be obtained in a noninvasive manner. Coherence gating is used to select minimum backscattered photons for image reconstruction. Axial and lateral resolutions are determined by the source coherence length and numerical aperture of the sampling lens, respectively. Suitability for grafting is based on minimal, and preferably the absence of, injury to the inside of the conduit wall, such as that which occurs during harvesting of the conduit (e.g., intimal tears and dissections). Suitability also can be based on minimal, and preferably die absence of, pre-existing pathology, such as intraluminal blood clots, clot strands, neointimal hyperplasia, and soft or hard plaques. Normal RA has a three-handed appearance (i.e., the intima, the media, and the adventitia of the internal elastic lamina (IEL)) on OCT scans, whereas normal SV has a two-banded appearance on OCT scans, since it lacks a clearly distinct intima layer. Intimal thickenings occur as eccentric abnormalities within RA and as more diffuse, concentric patterns within SV. Lipid-laden plaques appear as dark cores within a bright thick layer corresponding to the intima, whereas calcified plaques appear as shadows. The objective of such examination is to minimize, and, to the extent possible, eliminate, the likelihood of premature stenosis after transplantation. In addition, such examination informs the surgeon of his graft harvesting technique.

In view of the above, a method of examining a candidate conduit prior to harvesting for CABG is also provided. The method comprises using OCT to identify a portion of the candidate conduit as suitable for harvesting and subsequent grafting, whereupon the candidate conduit is examined prior to harvesting for CABG. Examples of candidate conduits include, but are not limited to, the intermamillary artery (IMA) in the chest wall, the RA in the forearm, and the SV in die leg. Typically, the MA does not require screening because it is only redirected—not harvested. Suitability of a candidate conduit for harvesting and subsequent grafting can be based on markers of premature graft failure, such as pre-existing plaque in the RA or intimal hyperplasia in the SV. The method can further comprise grafting the conduit into a patient in need of CABG.

Also provided is a method of assessing the quality of an anastomosis. The method comprises using OCT and an imaging catheter to generate serial images of the anastomosis from inside the anastomosis, generating a three-dimensional reconstruction of the anastomosis, examining the three-dimensional reconstruction of the anastomosis to assess the quality of the anastomosis, repairing the anastomosis as needed, and then closing the surgical incision used to suture the anastomosis, whereupon the quality of the anastomosis is assessed.

Further provided is a method of assessing the quality of the suturing of a small vessel or duct. The method comprises using OCT and an imaging catheter to generate serial images of the site of suturing from inside the small vessel or duct, generating a three-dimensional reconstruction of the site, examining the three-dimensional reconstruction of the site to assess the quality of the suturing, and repairing the suturing as needed, whereupon the quality of the suturing is assessed. The suture is coated with a contrast agent prior to suturing. In this regard, any suitable contrast agent cast be used. Desirably, the contrast agent is safe for use in the body and does not adversely affect the healing of the suturing. An example of a suitable contrast agent is gold, which is preferably applied as a very thin layer to the sutures prior to suturing of the small vessel or duct.

Still further provided is a method of detecting coronary artery disease in a harvested donor heart prior to transplantation. The method comprises scanning the harvested donor heart with OCT to general serial images of the heart and examining the images for signs of disease, whereupon coronary artery disease can be detected in a harvested donor heart prior to transplantation. Preferably, the harvested donor heart is placed in a clear, crystalloid solution prior to scanning with OCT. If plaques are detected, such a method enables stenting of plaques prior to transplantation. The benefits of such prophylactic treatment are the inhibition or prevention of further progression of the plaques after transplantation and the avoidance of an invasive stenting procedure in the transplant recipient.

### EXAMPLE

The following example is provided for the purpose of illustration. The example is not intended to limit the scope of the claimed invention.

### Example 1

This example demonstrates the reproducibility and accuracy of the method.

Conduits were procured endoscopically (37 SV and 8 RA) and by open technique (9 RA) from 50 patients. Surplus segments were analyzed by OCT for evidence of pre-existing pathology or traumatic injury. Focal plaques in RA and areas of intimal hyperplasia in SV were quantified as intimal/medial thickness ratio (IMT>0.5). Biopsies were obtained for histological confirmation and to analyze matrix metalloproteinase-2 levels (for SV) and prostacyclin/NO metabolites (for RA). Inter-observer kappa coefficients and a Bland-Altman analysis were used to determine the reproducibility and accuracy of OCT interpretations.

Imaging revealed plaque in 76% of the RA. Endoscopically harvested conduits showed intraluminal clots (38%) and intimal tears ranging from severe (6%) to mild (88%). Intimal thickening was detected in 86% of SV, whereas intraluminal clot was detected in 68% of SV, IMT as determined by OCT correlated significantly with matrix metalloproteinase-2 levels (R=0.6804) in SV and with metabolites of prostacyclin (R=−0.55) and NO (R=−0.58) in RA. OCT imaging was reproducible (inter-observer kappa coefficients>0.81 for the characterization of plaque types) and correlated strongly with histology (R=,8, p<0.001).

All references, including publications, patent applications, and patents, cited herein are hereby incorporated by reference to the same extent as if each reference were individually and specifically indicated to be incorporate by reference and were set forth in its entirety herein.

The use of the terms “a,” “an,” “the,” and similar referents in the context of describing the invention (especially in the context of the following claims) ate to be construed to cover both the singular and the plural, unless otherwise indicated herein or clearly contradicted by context. The recitation of ranges of values herein is merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to illustrate better the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. It should be understood dial the illustrated embodiments are exemplary only, and should not be taken as limiting the scope of the invention.

1-7. (canceled)

8. A method comprising:
identifying a portion of a conduit as being suitable for grafting, by examining the conduit using optical coherence tomography (OCT).

9. A graft comprising
a portion of a harvested conduit,
wherein the portion of the harvested conduit is selected as being suitable for grafting by examining the harvested conduit using optical coherence tomography (OCT) according to the method of claim 8.

10. The method of claim 8, wherein the conduit is examined using optical coherence tomography (OCT) prior to harvesting the conduit.

11. The method of claim 8, further comprising:
grafting the identified portion of the conduit into a patient in need of coronary artery bypass grafting.

12. A graft comprising
a portion of a conduit,
wherein the portion of the conduit is selected as being suitable for harvesting and subsequent grafting by examining the conduit using optical coherence tomography (OCT) prior to harvesting according to the method of claim 10.

13. A method comprising:
(i) generating serial images of an anastomosis or site of suturing a small vessel or duct from inside the anastomosis or small vessel or duct using optical coherence tomography (OCT) and an imaging catheter,
(ii) generating a three-dimensional reconstruction of the anastomosis or site from the serial images,
(iii) examining the three dimensional reconstruction of the anastomosis or site to assess the quality of the anastomosis or suturing, and
(iv) repairing the anastomosis or suturing as needed.

14. A three-dimensional reconstruction of an anastomosis generated from serial images of the anastomosis from inside the anastomosis, wherein the serial images are generated using optical coherence tomography (OCT) and an imaging catheter according to the method of claim 13.

15. A three-dimensional reconstruction of a site of suturing a small vessel or duct from inside the vessel or duct, wherein the three-dimensional reconstruction is generated from serial images of the suturing site from inside the small vessel or duct, wherein the serial images are generated using optical coherence tomography (OCT) and an imaging catheter according to the method of claim 13.

16. A method of detecting coronary artery disease in a harvested donor heart prior to transplantation, comprising scanning the harvested donor heart with optical coherence tomography (OCT) to generate serial images of the heart and examining the images for signs of disease, wherein coronary artery disease can be detected in a harvested donor heart prior to transplantation.

17. The method of claim 16, wherein the harvested donor heart is placed in a clear, crystalloid solution prior to scanning with OCT.

18. Serial images of a harvested donor heart, wherein the serial images of the donor heart are generated using optical coherence tomography on the harvested donor heart before transplantation according to the method of claim 16.
